(19) United States
(12) Patent Application Publication
(10) Pub. No.: US 2007/0015165 A1
(43) Pub. Date: Jan. 18, 2007

(54) Method for the isolation of RNA from biological sources

(75) Inventors: Fuqiang Chen, St. Louis, MO (US); David Cutter, St. Louis, MO (US)

Correspondence Address: SENNIGER POWERS, ONE METROPOLITAN SQUARE, 16TH FLOOR, ST LOUIS, MO, 63102, US

(73) Assignee: Sigma-Aldrich Co., St. Louis, MO (US)

(21) Appl. No.: 11/180,087

(22) Filed: Jul. 13, 2005

(51) Int. Cl. C12Q 1/68; C07H 21/02

(52) U.S. Cl. 435/6; 435/270; 536/25.4

(57) ABSTRACT

Methods and kits for isolating RNA are provided that enable rapid RNA preparation from biological sources. In one aspect, RNA is isolated from difficult plant tissues and cells that contain high levels of secondary metabolites, without employing organic extraction or salt precipitation procedures. This method employs novel lysing and binding conditions to allow preparation of RNA free from secondary metabolites.

### FIELD OF THE INVENTION

The present invention generally relates to a process for purifying RNA from biological sources containing nucleic acids. In particular, the present invention relates to a process for purifying RNA from plant samples.

### BACKGROUND OF THE INVENTION

The nucleic acids, deoxyribonucleic acid (DNA) and ribonucleic acid (RNA) are found in all living cells. DNA is the genetic material and genes are transcribed into messenger RNA (mRNA) which is then translated into protein. In addition to mRNA, the other major types of RNA are transfer RNA (tRNA) and ribosomal RNA (rRNA). Analysis of gene expression through the study of mRNA is of fundamental importance in the field of life science. mRNA levels are studied by a variety of techniques including polymerase chain reaction (PCR), quantitative polymerase chain reaction (qPCR), northern blotting, and microarrays. In all of these techniques it is necessary to purify the mRNA free of contaminants found in living cells that include genomic DNA, proteins, lipids, phenolic compounds, polysaccharides, and other biomolecules.

Purification of RNA from biological sources entails isolating RNA from DNA as well as from other components making up biological samples. In many plant species and tissues, hereinafter referred to as difficult plant tissues and cells, secondary metabolites, such as phenolic compounds, and polysaccharides, often interfere with RNA isolation and its use in downstream techniques. These secondary plant metabolites can impair RNA purification and/or degrade RNA thereby hindering gene expression analysis. Additionally, mRNA is normally degraded within minutes by ribonucleases that are present within plant cells. Therefore, for purification of mRNA it is critical that the procedure be fast and that ribonucleases are inactivated. It is also important when purifying RNA from difficult plant tissues and cells that steps are taken to prevent interference from secondary metabolites. As a consequence, laborious procedures as well as extraction with hazardous organic solvents, such as phenol and chloroform, are often required in methods of the known art to prepare RNA from such plant tissues.

Methods for purification of nucleic acids can be broadly classified within three categories: differential solubility, adsorption chromatography, and centrifugation methods. Differential solubility makes use of extraction in organic solvents such as phenol and chloroform. Adsorption chromatography binds nucleic acids to a matrix in the presence of chaotropic agents. Centrifugation methods include differential centrifugation and density gradient centrifugation.

The current state of the art in isolating RNA from plant tissues with high concentrations of phenolics and polysaccharides involves lysing cells in a buffer containing various agents, such as detergent, borate, PEG, PVP, and PVPP. But invariably, crude RNA has to be precipitated from the extract and then purified with at least one round of phenol and chloroform extraction. In addition, differential precipitation by lengthy centrifugation in high salt is often required for removing polysaccharides (Kolosova et al., 2004; Pateraki and Kanellis, 2004). Thus, the purification process is time consuming and laborious, and involves hazardous organic solvents.

Many commercial RNA purification kits, though providing a rapid procedure for some plant tissues, are often totally ineffective for difficult tissues containing phenolic compounds or polysaccharides. U.S. Pat. No. 6,875,857 reveals a method that employs a high volume of 2-mercaptoethanol (up to 40% volume), a non-ionic detergent, and an anionic detergent for overcoming interfering secondary metabolites. But again, RNA has to be further purified by chloroform extraction and alcohol precipitation. The method, while representing an improvement, is still time consuming and involves chloroform. Moreover, a high volume of 2-mercaptoethanol is malodorous and hazardous. Therefore, there is presently a need in the art for a better RNA purification method that is suitable for a wide range of plant tissues, including those enriched in interfering secondary metabolites, without using hazardous organic solvents.

In addition, present methods for purification of RNA from mammalian cells often result in RNA that is contaminated with genomic DNA. Therefore, there is a need in the art for a method that is fast, that does not involve the use of organic solvents and result in high purity RNA that is essentially free of contaminating genomic DNA.

### SUMMARY OF THE INVENTION

One aspect of the invention is a method of purifying RNA from a biological sample containing difficult plant tissues or cells that contain high levels of phenolic compounds and/or polysaccharides. In another aspect of the present invention, a method of purifying RNA is provided wherein RNA is purified from a biological sample without the use of phenols or chloroform. In still another aspect, the present invention provides a rapid method of purifying RNA from a biological sample.

Briefly, therefore, the present invention is directed to a method of isolating RNA from a biological sample. The method comprises lysing the biological sample with a solution comprising a chaotrope and a detergent to release RNA into the solution. The released RNA is bound to a matrix in the presence of a monovalent salt wherein the concentration of the monovalent salt in the RNA-containing solution is at least about 3 M.

The present invention is also directed to a method for isolating RNA from a solution. The method comprising binding RNA to a matrix in the presence of a monovalent salt wherein the concentration of the monovalent salt in the RNA-containing solution is at least about 3 M, and separating the solution from the bound RNA.

The present invention is also directed to a reagent for isolating RNA from difficult plant tissues or cells, said reagent comprising a detergent, a chaotrope, a chelator, and a reducing agent.

The present invention is also directed to a kit for isolating RNA from a biological sample. The kit comprises a reagent and a binding solution. The reagent is comprised of a chaotrope, a detergent, and a chealtor. The binding solution comprises at least about 3 M LiCl.

The present invention is also directed to a reagent for isolating RNA from plant tissues. The reagent comprises a detergent selected from the group consisting of nonionic polyoxyethylenes and cationic quaternary ammonium compounds; guanidine hydrochloride; and at least about 3 M LiCl.

Other aspects and features of this invention will be in part apparent and in part pointed out hereinafter.

### BRIEF DESCRIPTION OF THE DRAWINGS

FIG. 1. is an illustration of an electrophoresis of RNA samples from Norway Spruce wherein RNA is purified with and without detergent.

FIG. 2. is an illustration of an electrophoresis of RNA samples from pine needles wherein RNA is purified with and without detergent.

FIG. 3 is a bar graph illustrating the effect of LiCl concentration on RNA adsorption from pine needles.

FIG. 4 is a bar graph illustrating the effect of LiCl concentration on RNA adsorption from corn leaves.

FIG. 5 is an illustration of an electrophoresis comparing the effects of different guanidine salts on RNA purification from different plant tissues.

FIG. 6 is an illustration of an electrophoresis comparing the effects of 2-mercaptoethanol on RNA purification from plant tissues.

FIG. 7 is a bar graph comparing RNA binding to silica matrix with and without chaotrope.

FIG. 8 is a line graph comparing monovalent salts on RNA binding to silica matrix.

FIG. 9 is a bar graph comparing RNA binding on siliceous and non-siliceous matrices.

### DETAILED DESCRIPTION OF THE INVENTION

The present invention relates to an improved process of purifying RNA from plant or animal biological sources containing nucleic acid. Additionally, the process of the present invention provides a more rapid method in which RNA may be purified from biological sources containing secondary metabolites. In particular, the invention relates to a process of purifying RNA from a biological sample containing nucleic acid without utilizing phenol or chloroform extraction reagents.

Difficult plant tissues or cells, for the purposes of the present invention, are plant tissues or cells that contain high levels of secondary metabolites such as polysaccharides, phenolic compounds, polyphenolic compounds, and/or tannins. Concentrations of secondary metabolites can vary greatly from species to species, tissue to tissue, growth stage to growth stage, and from environment to environment. In one embodiment, difficult plant tissues or cells comprise at least about 5% polysaccharides. In another embodiment, difficult plant tissues or cells comprise at least about 0.05% phenolic or polyphenolic compounds. In still another embodiment, difficult plant tissues or cells comprise at least about 0.1% phenolic or polyphenolic compounds. Examples of difficult plant tissues that contain high levels of phenolic compounds and polyphenolic compounds include, but are not limited to, gymnosperm conifer needles, cotton leaves, red maple leaves, and grape leaves. Examples of plant tissues that contain high levels of polysaccharides include, but are not limited to, seeds, fruit, tubers, and plant tissues that are under environmental stresses. Specific examples of such plant tissues include potato tuber, sweet potato tuber, cassava tuber, corn kernel, and other cereal grains. While appreciable concentrations of either phenolic compounds or polysaccharides can reduce yield of RNA that can be isolated from difficult plant tissues or cells, the mechanisms that reduce RNA yields differ.

It is not always apparent which secondary metabolite compounds, if any, are present in plant tissue samples. Since phenolic compounds and polysaccharides are ubiquitous in plants, it is not uncommon that a plant tissue sample contains high concentrations of both phenolic compounds and polysaccharides. In some cases, the secondary metabolites are primarily phenolic compounds. In other instances, the secondary metabolites are polysaccharides. The methods of the present invention can be utilized to isolate RNA from difficult plant tissues or cells that contain secondary metabolites.

In one embodiment, the present invention relates to an RNA isolation method that enables rapid RNA isolation from difficult plant tissues or cells that contain high levels of phenolic compounds or polysaccharide secondary metabolites, without employing organic extraction or salt precipitation procedures that are common in the art.

Phenolic compounds can reduce RNA yield by directly damaging RNA and other nucleic acids that are present in a sample through oxidative or cross-linking reactions. Conventional methods of isolating RNA from biological tissue that contains high concentrations of phenolic compounds require time-consuming steps and the use of hazardous organic solvents, such as phenol and chloroform.

Polysaccharides, in contrast, can reduce RNA yield by interfering with the purification process and reduce RNA yield and quality if not removed. In conventional silica chromatographic systems and methods for isolating RNA, alcohol, typically 70%-100% ethanol, is added to biological extracts in the presence of a chaotrope to promote the binding of RNA to a matrix. However, polysaccharides and genomic DNA often precipitate out of biological extracts and form aggregates when alcohol is introduced in preparation for RNA adsorption. These aggregates can clog the matrix surface to which the RNA binds, thereby reducing the selectivity of RNA adsorption resulting in poor RNA yield and quality of the isolated RNA as well as contamination of RNA with genomic DNA. In order to prevent polysaccharide aggregates, conventional methods of isolating nucleic acid remove polysaccharides through time-consuming precipitation processes utilizing high salt differentials.

The present invention, in contrast to prior art methods that are both time-consuming and require utilizing hazardous organic solvents, provides greatly simplified methods for isolating RNA from difficult plant tissues or cells. In one aspect of the present invention, a combination of a chaotrope and a detergent are mixed to form a lysis solution. The lysis solution, when mixed with a biological sample, inactivates ribonucleases and reduces the damaging effects of phenolic compounds. The mixture of the lysis solution and biological sample is then contacted with a matrix in the presence of a monovalent salt without initiating aggregation of polysaccharides and genomic DNA. The RNA contained in the mixture binds to the matrix, thereby being isolated from the other cellular constituents. The RNA is then eluted from the matrix and recovered.

The purpose of the chaotrope is to disrupt molecular interactions and to deactivate ribonuclease present in the biological sample. The molecular interactions that may be disrupted include disrupting bonds other than covalent bonds, such as hydrogen bonds and electrostatic bonds. In one embodiment, the chaotrope concentration in the lysis solution is at least about 0.5 M. In another embodiment, the chaotrope concentration is at least about 4 M. In another embodiment, the chaotrope concentration is between about 5 M and about 7M. In still another embodiment, the chaotrope concentration is between about 5 M and about 6 M.

Chaotropes that may be used in the process of the present invention can include, but are not limited to, guanidine hydrochloride (guanidine HCl), sodium perchlorate, and urea. In one embodiment, the lysis solution contains guanidine hydrochloride.

Incorporating detergents in the lysis solution can beneficially result in an increased recovery of RNA in difficult plant tissues or cells that contain phenolic compounds. By incorporating a detergent in the lysis solution, RNA can be isolated in high quality and high yields without requiring additional extraction steps.

As discussed further in the examples below, RNA cannot be isolated from some difficult plant tissues or cells without incorporating a detergent in the lysis solution. It has been determined from experiments utilizing methods for isolating RNA from difficult plant tissues or cells containing phenolic compounds, that RNA partitions to the solid debris when no detergent is present in the lysis solution. Without a detergent in the lysis solution, the RNA recoverable from the lysate supernatant either by alcohol precipitation or by detergent rescues is reduced. However, by re-extracting the RNA-containing solid cellular debris with a lysis solution containing a detergent, a fraction of partially degraded RNA is able to be recovered.

The exact mechanism by which the detergents facilitate the isolation of RNA in plant tissues containing high levels of phenolic compounds is not known. However, without being held to any particular theory, it is believed that an effective detergent, when incorporated in the lysis solution of the present invention, has a high affinity for phenolic compounds and other damaging secondary metabolites. The detergent interacts with these compounds to form complex micelles, effectively preventing the compounds from damaging nucleic acids through oxidization and/or cross-linking reactions. It is also possible, however, that different classes of detergents may function by different mechanisms.

The method of the present invention beneficially integrates a detergent and a chaotropic agent in a lysis solution of a matrix adsorption system wherein the removal of damaging secondary metabolites and the isolation of RNA take place simultaneously without requiring additional extraction/isolation steps or reagents. Thus, unlike prior art methods which can require several hours to isolate RNA after the plant tissue has been ground, the methods of the present invention for isolating RNA can require less than thirty minutes. Furthermore, the methods of the present invention isolate RNA from difficult plant tissues or cells without requiring the use of hazardous organic solvents such as phenol and chloroform.

Detergents that can be used in the process of the present invention can include, but are not limited to, Igepal CA-630 (Sigma-Aldrich, St. Louis, Mo.), Tween 20 (Sigma-Aldrich, St. Louis, Mo.), polyoxyethylene detergents, quaternary ammonium compounds, and polyvinylpyrrolidone. Polyoxyethylenes are non-ionic detergents, while quaternary ammonium compounds are cationic detergents. Non-limiting examples of polyoxyethylenes that can be used in the present invention include polyoxyethylenesorbitan monolaurate (Tween 20, Sigma-Aldrich, St. Louis, Mo.), polyoxyethylenesorbitan monooleate (Tween 80, Sigma-Aldrich, St. Louis, Mo.), octylphenoxy poly(ethyleneoxy)ethanol (Igepal CA 630, Sigma-Aldrich, St. Louis, Mo.), and t-octylphenoxypolyethoxyethanol (Triton X100 and Triton X114, Sigma-Aldrich, St. Louis, Mo.), and Nonidet P-40 (NP-40, Sigma-Aldrich, St. Louis, Mo.). Non-limiting examples of quaternary ammonium compounds include hexadecyltrimethylammonium bromide (CTAB, Sigma-Aldrich, St. Louis, Mo.), dodecyltrimethylammonium bromide, ethylhexadecyldimethylammonium bromide, benzethonium chloride (Hyamine 1622, Sigma-Aldrich, St. Louis, Mo.), and benzyldimethylhexadecylammonium chloride. The detergents may be incorporated in the lysis solution alone or as a combination of two or more detergents.

Polyvinylpyrrolidone (PVP-40), which is not a detergent, also exhibits a slight effect in improving RNA isolation when incorporated in the lysis solution.

In one embodiment, the detergent concentration in the lysis solution is between about 0.1% to about 10%. In another embodiment, the detergent concentration is between about 1% and 5%. In still another embodiment, the detergent concentration is between about 1% and 2%.

A monovalent salt is utilized to promote adsorption of the RNA in the biological sample to the surface of a matrix without inducing the aggregation of polysaccharides or genomic DNA. Monovalent salts which may be used include, but are not limited to, lithium chloride (LiCl), lithium acetate, and ammonium acetate. When LiCl is utilized, however, the chaotrope utilized in the lysis solution must be a compound other than guanidine thiocyanate, such as guanidine hydrochloride.

In one embodiment, the monovalent salt is contained in the lysis solution. The use of a monovalent salt in the lysis solution causes the RNA contained in the biological sample to bind to the matrix upon contact. Thus, in one embodiment, the biological sample is mixed with the lysis solution and centrifuged with a matrix for about three minutes or less. In another embodiment, the biological sample and lysis solution mixture is centrifuged with a matrix for about one minute or less.

In another embodiment, the monovalent salt is contained in a binding solution that is mixed with an RNA-containing solution.

While conventional methods of nucleic acid isolation utilize LiCl in concentrations of 2 M or less for precipitation purposes, the present invention utilizes a higher concentration of monovalent salt in an RNA-containing solution to promote adsorption of the RNA to the surface of a matrix. In one embodiment, the monovalent salt concentration in the RNA-containing solution is at least about 3 M. In another embodiment, the monovalent salt concentration is at least about 5 M. In another embodiment, the monovalent salt concentration is at least about 10 M. In another embodiment, the monovalent salt concentration is between about 5 M and about 14M. In another embodiment, the monovalent salt concentration is between about 11 M and about 13 M. In still another embodiment, the monovalent salt concentration is about 12 M.

In another embodiment, a binding solution comprising a mixture of a monovalent salt and an alcohol are used to bind the RNA to a matrix. Non-limiting examples of alcohols that can be used in the present invention include ethanol and isopropyl alcohol. For example, a binding solution mixture containing half ethanol and half of a 12 M solution of LiCl can be used to isolate RNA from difficult plant tissues or cells. While RNA is recovered in high yields and high quality when the binding solution contains a monovalent salt but does not contain an alcohol, a binding solution containing an alcohol and a monovalent salt still provides superior isolation of RNA compared to a binding solution containing an alcohol and no monovalent salt.

In another embodiment, the binding solution can contain both an alcohol and a monovalent salt when isolating RNA from difficult plant tissues or cells.

The matrix used in the present invention may be any solid matrix to which RNA can be bound. In one embodiment, the matrix can comprise a hydrophilic matrix. The hydrophilic matrix can be comprised of an organic binding matrix or an inorganic binding matrix. Non-limiting examples of organic binding matrices include acrylic copolymer, cellulose, dextran, agarose, and acrylic amide. Non-limiting examples of inorganic binding matrices include silica, diatomaceous earth, aluminum oxides, glass, titanium oxides, zirconium oxides, and hydroxyapatite. Examples of silica matrices include, but are not limited to, silica particles, silica filters, magnetized silica, and the like.

In addition to a chaotrope and a detergent, the lysis solution can also be formulated to contain chelators to enhance the beneficial effects of the detergent. Examples of chelators can include, but are not limited to, ethylenediaminetetraacetic acid (EDTA), ethylene glycol bis(2-aminoethyl ether)-N,N,N′N′-tetraacetic acid (EGTA), and cyclohexane-trans-1,2-diamine tetraacetic acid (CDTA).

The pH of the lysis solution also beneficially enhances the effect of the detergent. In one embodiment, the pH of the lysis solution is above pH 6. In another embodiment, the pH of the lysis solution is above pH 6 and less than or equal to about pH 8.5. In still another embodiment, the pH is between about pH 7 and about pH 8.

The lysis solution can also be supplemented with a reducing agent. Examples of reducing agents that can be used in the present invention include, but are not limited to, 2-mercaptoethanol and dithiothreitol (DTT). While not required, the reducing agent can improve RNA recovery and quality in tissues that contain high levels of ribonucleases. In one embodiment, the reducing agent 2-mercaptoethanol is incorporated in the lysis solution at a concentration between about 1% and about 5%. In another embodiment, the concentration of 2-mercaptoethanol in the lysis solution is between about 1% and about 2%.

The lysis solution, in addition to containing a chaotrope and a detergent, can also comprise a monovalent salt. In one embodiment, the lysis solution for isolating RNA from plant tissues comprises guanidine hydrochloride, a detergent, and LiCl.

The lysis solution can be formulated to contain a chaotrope, a detergent, a monovalent salt, and a matrix. In one embodiment, the lysis solution for isolating RNA from plant tissues comprises guanidine hydrochloride, a detergent, LiCl, and a matrix containing a porous silica surface.

The lysis solution can also be formulated to contain a chaotrope, a detergent, a monovalent salt, a matrix, and a chelator. In one embodiment, the lysis solution comprises guanidine hydrochloride, a detergent, LiCl, a matrix containing a porous silica surface, and EDTA.

In still another embodiment, the lysis solution comprises guanidine hydrochloride, a detergent, LiCl, a matrix containing a porous silica surface, and EDTA, wherein the lysis solution has a pH between about 7 and about 8.

Once the RNA is bound to the matrix, the matrix can be optionally washed. In one embodiment, the matrix is optionally washed with a salt solution. Non-limiting examples of salt solutions include LiCl, guanidine thiocyanate, and guanidine hydrochloride salt solutions. In another embodiment, the matrix is washed with an alcohol wash solution. Examples of alcohol wash solutions include, but are not limited to, ethanol and isopropanol wash solutions.

The bound RNA is recovered and isolated by elution from the matrix. In one embodiment, the RNA is eluted from the matrix with an RNase-free low salt solution that contains less than about 50 mM of salt. The RNase-free low salt solution can comprise 10 mM Tris, 1 mM EDTA, pH 7-8. In another embodiment, the RNA is eluted from the matrix by washing the matrix with RNase-free water.

An exemplary method for isolating RNA from a plant tissue sample is described as follows:

1) Grind plant tissues into a fine powder in liquid nitrogen.

2) Add 500 μl of a lysis solution (supplemented with 2-mercaptoethanol at 10 μl/ml lysis solution) to approximately 100 mg of the plant tissue sample in a micro-centrifuge tube. Vortex the tube immediately and vigorously and incubate the vortexed mixture at 55° C. for three minutes. An exemplary lysis solution is comprised of 6 M guanidine hydrochloride, 50 mM Tris-HCl (pH 7.0), 90 mM EDTA (pH 8.0), and 1.5% (v/v) Tween 20 wherein the final solution has a pH of 7.5.

3) Centrifuge the sample for three (3) minutes at maximum speed (e.g., 12,000×g) at room temperature to pellet the cellular debris. Transfer the lysate supernatant onto a filtration column (Sigma Product Code G6415, Sigma-Aldrich, St. Louis, Mo.) seated in a 2 ml collection tube and centrifuge for one minute at maximum speed to remove residual cellular debris.

4) Add 250 μl of a 12 M LiCl solution to the flow-through cleared lysate and mix by brief vortex or pipetting five times. Transfer the mixture into a binding column (Sigma Product Code G4669, Sigma-Aldrich, St. Louis, Mo.) and centrifuge for one minute at maximum speed to bind RNA. Decant flow-through liquid. For tissues that contain a high water content, such as root, fruit, and succulent tissues, the amount of 12 M LiCl solution is increased to 500 μl.

5) Wash the column with 500 μl of a 2 M LiCl solution and centrifuge for one minute to remove residual genomic DNA.

6) Wash the column with 500 μl of an alcohol solution (10 mM Tris-HCl, pH 7.0, 80% ethanol) and centrifuge for 30 seconds. Decant the flow-through fluid and repeat the wash once. Dry the column with one minute of centrifugation.

7) Transfer the column to a new 2-ml collection tube and add 50 μl of RNase-free water directly onto the filter surface inside the column. Centrifuge for one minute to elute RNA.

In another embodiment, the present invention relates to an improved process for purifying RNA from biological sources that do not contain high levels of polysaccharides. For example, purifying RNA from biological sources containing less than about 5% soluble polysaccharides. In this embodiment, the lysis solution comprises a chaotrope and a detergent. The RNA is isolated on a matrix in the presence of either a monovalent salt, an alcohol, or mixture thereof, wherein the monovalent salt concentration in the RNA-containing solution is at least about 3 M. In another embodiment, the monovalent salt concentration in the solution is at least about 5 M. In still another embodiment, the monovalent salt concentration in the solution is about 12 M.

In another aspect of the present invention, the methods of the present invention can be utilized to isolate RNA from plant tissue that does not contain high concentrations of phenolic compounds. For example, purifying RNA from biological sources containing less than about 0.1% phenolic compounds. In this embodiment, use of a plant tissue sample that does not contain high concentrations of phenolic compounds is mixed with a lysis solution containing a chaotrope. RNA from the tissue sample is isolated on a matrix in the presence of a monovalent salt, wherein the monovalent salt concentration in the solution is at least about 3 M. In another embodiment, the monovalent salt concentration in the solution is at least about 5 M. In still another embodiment, the monovalent salt concentration in the solution is about 12 M.

The methods of the present invention can also be utilized to isolate RNA from animal tissues or cells. In one embodiment, the lysis solution for isolating RNA from animal tissues or cells comprises a chaotrope and a detergent. The animal tissue is mixed with the lysis solution and the RNA is bound to a matrix in the presence of a monovalent salt, wherein the monovalent salt concentration in the RNA-containing solution is at least about 3 M. In another embodiment, the monovalent salt concentration is at least about 5 M. In still another embodiment, the monovalent salt concentration is about 12 M.

In another embodiment, the lysis solution for isolating RNA from animal tissues or cells comprises a guanidine hydrochloride and a detergent. The animal tissue is mixed with the lysis solution and the RNA is bound to a porous silica surface in the presence of LiCl, wherein the concentration of LiCl in the RNA-containing solution is at least about 3M.

In another aspect of the present invention, RNA can be isolated from an RNA-containing solution, for example, an RNA-containing solution resulting from enzymatic reactions. The RNA contained in the solution has already been released from tissues and cellular components into the solution. In one embodiment, the RNA-containing solution is contacted with a matrix in the presence of a monovalent salt, wherein the monovalent salt concentration in the solution is at least about 3 M. In another embodiment, the monovalent salt concentration in the solution is at least about 5 M. In still another embodiment, the monovalent salt concentration in the solution is about 12 M.

In another embodiment, the pH of the RNA-containing solution is greater than 6. In another embodiment, the pH of the RNA-containing solution is about 7 or above. In another embodiment, the RNA-containing solution is contacted with a matrix in the presence of a monovalent salt wherein the pH is between about 7 and about 8.5. In still another embodiment, the RNA-containing solution is contacted with a matrix in the presence of a monovalent salt wherein the pH is between about 7 and about 8.

In another embodiment, the RNA-containing solution is contacted with a matrix in the presence of a monovalent salt and a chaotrope.

In another embodiment, the RNA is isolated from an RNA-containing solution in the absence of a chaotrope.

In still another embodiment, the RNA is isolated from an RNA-containing solution in the absence of a detergent.

Kits

In one embodiment, the present invention comprises a kit comprising a reagent for isolating RNA from plant tissues. The kit can comprise one or more of the following components: a reagent for isolating RNA from a biological sample; a nucleic acid binding matrix; a filtration column; a binding solution; a salt wash solution; an alcohol wash solution; and a collection tube.

The reagent contains a detergent and a chaotrope. In one embodiment, the reagent contains a detergent that is selected from the group of nonionic polyoxylethylenes and cationic quaternary ammonium compounds. In another embodiment, the chaotrope in the reagent comprises guanidine hydrochloride. In another embodiment, the reagent further contains a chelator wherein the chelator is selected from EDTA, EGTA, or CDTA. In another embodiment, the reagent further contains a reducing agent wherein the reducing agent is selected from 2-mercaptoethanol or dithiothreitol (DTT). In still another embodiment, the reagent further contains a detergent selected from the group of nonionic polyoxyethylenes and cationic quaternary ammonium compounds; guanidine hydrochloride; a chelator wherein the chelator is selected from EDTA, EGTA, or CDTA; and a reducing agent wherein the reducing agent is selected from 2-mercaptoethanol or DTT. In another embodiment, the reagent further contains a monovalent salt selected from lithium chloride, lithium acetate, or ammonium acetate.

In one embodiment, the kit includes a reagent comprising a chaotrope, a detergent, and a chelator; and a binding solution comprising at least about 3 M LiCl.

In another embodiment, the present invention comprises a reagent containing a chaotrope, a detergent, a chelator, and a reducing agent; a nucleic acid binding matrix; a filtration column; and a binding solution for isolating RNA from a biological sample. In one embodiment, the binding matrix is selected from a hydrophilic matrix. Examples of hydrophilic matrices include silica, diatomaceous earth, aluminum oxides, glass, titanium oxides, zirconium oxides, and hydroxyapatite. In another embodiment, the binding solution contains an alcohol selected from ethanol or isopropanol. In another embodiment, the binding solution contains a monovalent salt selected from lithium chloride, lithium acetate, or ammonium acetate. In still another embodiment, the reagent contains a monovalent salt selected from lithium chloride, lithium acetate, or ammonium acetate.

The following examples further illustrate the invention.

### EXAMPLE 1

### RNA Purification from Norway Spruce and Pine Needles

RNA Purification from Norway Spruce and Pine Needles with and without Detergents

Norway Spruce and pine needles were harvested and ground to a fine powder in liquid nitrogen. For each RNA extraction, 100 mg of the powdered plant material was lysed at 56° C. for 3 minutes in 450 μl of one of the three lysis solutions: 1) 6 M guanidine hydrochloride, 50 mM Tris-HCl, 95 mM EDTA, 1% 2-mercaptoethanol, pH 7.8; 2) 6 M guanidine hydrochloride, 50 mM Tris-HCl, 95 mM EDTA, 1% 2-mercaptoethanol, 1% Igepal CA-630, pH 7.8; 3) 6 M guanidine hydrochloride, 50 mM Tris-HCl, 95 mM EDTA, 1% 2-mercaptoethanol, 1% Tween 20, pH 7.8. The extract was filtered through a filtration column (Sigma Product Number C9346) by centrifugation for 2 minutes at 16,000×g to remove cellular debris. The clarified extract was mixed with a half volume of a 12 M LiCl binding solution, and the mixture was forced through a silica binding column by centrifugation for 1 minute at 16,000×g. The column was washed once with 700 μl of a salt wash solution (1 M guanidine thiocyanate, 12.5 mM Tris-HCl, 6.25 mM EDTA, pH 7.0) by centrifugation for 1 minute at 16,000×g, and then twice with 500 μl of an alcohol solution (80% ethanol, 10 mM Tris-HCl, pH 7.0) by centrifugation for 30 seconds at 16,000×g. After the column was dried by centrifugation for 1 minute at 16,000×g, bound RNA was eluted in 50 μl of RNase-free water by centrifugation for 1 minute at 16,000×g.

Comparative Example of RNA Purification from Norway Spruce and Pine Needles

A commercial kit (RNeasy Plant Mini Kit, Qiagen, Valencia, Calif.) was used to extract RNA. For each RNA extraction, 100 mg of the powdered plant material obtained as described in Example 1 was lysed using each of the two different lysis buffers provided in the kit (RLT, containing guanidine thiocyanate; and RLC, containing guanidine hydrochloride). RNA purification was carried out according to the kit's instruction.

Analysis of Purified RNA by Spectrophotometry and Agarose Gel Electrophoresis

Spectrophotometric Analysis of Norway Spruce needle:
(a) Lysis Solution #1 (no detergent) yielded no RNA.
(b) Lysis Solution #2 (containing 1% Igepal) yielded from 36 to 54 μg RNA per preparation, with A₂₆₀/A₂₈₀ ratio equal to 2.0.
(c) Lysis Solution #3 (containing 1% Tween 20) yielded from 42 to 51 μg RNA per preparation, with A₂₆₀/A₂₈₀ ratio equal to 2.0.
(d) RNeasy Plant Mini Kit with RLT buffer yielded no RNA.
(e) RNeasy Plant Mini Kit with RLC buffer yielded less than 4 μg of RNA per preparation, with A₂₆₀/A₂₈₀ ratio equal to 1.6.

Spectrophotometric Analysis of Pine Needles
(a) Lysis Solution #1 (no detergent) yielded no RNA.
(b) Lysis Solution #2 (containing 1% Igepal) yielded from 9 to 13 μg RNA per preparation, with A₂₆₀/A₂₈₀ ratios ranging from 1.9 to 2.0.
(c) Lysis Solution #3 (containing 1% Tween 20) yielded from 19 to 23 μg RNA per preparation, with A₂₆₀/A₂₈₀ ratios ranging from 1.9 to 2.0.
(d) RNeasy Plant Mini Kit (with RLT or RLC buffer) yielded no RNA.

Results of agarose gel electrophoresis: For each sample, 2 μl of eluate was analyzed in 1% nondenaturing agarose gel.

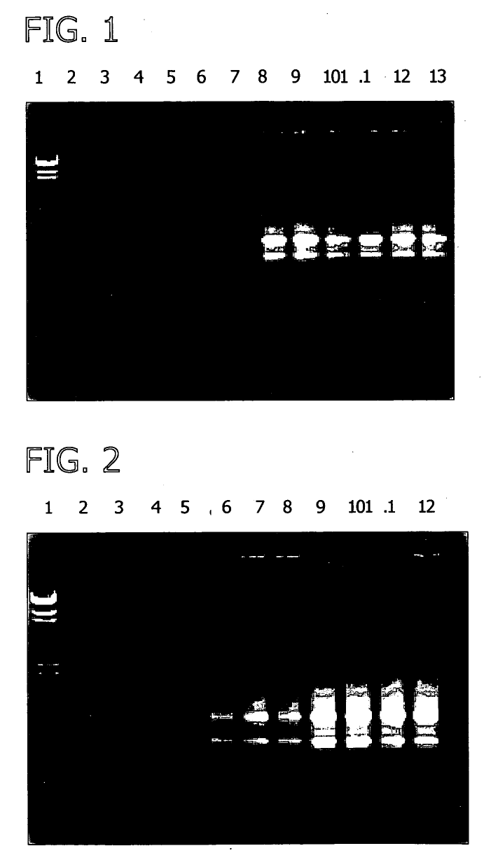

FIG. 1. is an illustration of an electrophoresis of RNA samples from Norway Spruce.

Lane 1: Lambda/Hind III DNA ladder; lanes 2-3: samples prepared by RNeasy Plant Mini Kit with RLT Buffer, lanes 4-5: samples prepared by RNeasy Plant Mini Kit with RLC Buffer; lanes 6-7: samples prepared with 6 M guanidine hydrochloride, 50 mM Tris-HCl, 95 mM EDTA, 1% 2-mercaptoethanol, pH 7.8 (Lysis Solution #1); lanes 8-11: samples prepared with 6 M guanidine hydrochloride, 50 mM Tris-HCl, 95 mM EDTA, 1% 2-mercaptoethanol, 1% Igepal CA-630, pH 7.8 (Lysis Solution #2); lanes 12-13: samples prepared with 6 M guanidine hydrochloride, 50 mM Tris-HCl, 95 mM EDTA, 1% 2-mercaptoethanol, 1% Tween 20, pH 7.8 (Lysis Solution #3).

FIG. 2. is an illustration of an electrophoresis of RNA samples from pine needles

Lane 1: Lambda/Hind III DNA ladder; lane 2: sample prepared by RNeasy Plant Mini Kit with RLT Buffer, lane 3: sample prepared by RNeasy Plant Mini Kit with RLC Buffer; lanes 4-5: samples prepared with 6 M guanidine hydrochloride, 50 mM Tris-HCl, 95 mM EDTA, 1% 2-mercaptoethanol, pH 7.8 (Lysis Solution #1); lanes 6-8: samples prepared with 6 M guanidine hydrochloride, 50 mM Tris-HCl, 95 mM EDTA, 1% 2-mercaptoethanol, 1% Igepal CA-630, pH 7.8 (Lysis Solution #2); lanes 9-12: samples prepared with 6 M guanidine hydrochloride, 50 mM Tris-HCl, 95 mM EDTA, 1% 2-mercaptoethanol, 1% Tween 20, pH 7.8 (Lysis Solution #3).

Example 1 Summary: This example illustrates the significance of a nonionic detergent in purifying RNA from the difficult plant materials, Norway spruce and pine needles.

### EXAMPLE 2

### Performance of Detergents in RNA Purification from Difficult Plant Tissues

Various detergents were evaluated for RNA extraction from pine needles and grape leaves. Plant tissue was ground to a fine powder in liquid nitrogen. For each test, 100 mg of powdered plant material was lysed at 56° C. for 3 minutes in 500 μl of a lysis solution comprising 6 M guanidine hydrochloride, 50 mM Tris-HCl, 90 mM EDTA, 1% 2-mercaptoethanol, pH 7.5, and 1.5% of one of the detergents listed in Table 1. Bulk cellular debris was removed by centrifugation for 3 minutes at 16,000×g. The supernatant extract was filtered through a filtration column (Sigma Number C6866) by centrifugation for 1 minute at 16,000×g to remove residual cellular debris. The clarified extract was then mixed with 250 μl of a 12 M LiCl binding solution. The mixture was forced through a binding column (Sigma product C6991) by centrifugation for 1 minute at 16,000×g. The column was washed once with 500 μL of a 2 M LiCl solution by centrifugation for 1 minute at 16,000×g, and then twice with 500 μl of an alcohol solution (80% ethanol, 10 mM Tris, pH 7.0) by centrifugation for 30 seconds at 16,000×g. After the column was dried by centrifugation for 1 minute at 16,000×g, bound RNA was eluted in 50 μl of RNase-free water by centrifugation for 1 minute at 16,000×g. Purified RNA was analyzed by a spectrophotometer and by agarose gel electrophoresis to determine the effectiveness of each detergent. The results are summarized in Table 1. Effective detergents were further verified with other well-known difficult plant tissues containing phenolic compounds, such as cotton leaf and red maple leaf. No RNA could be isolated from these tissues without an effective detergent (results not shown).

TABLE 1
The effectiveness of various detergents in RNA purification
from pine needles and grape leaves.

| | | Acronym/ | |
|---|---|---|---|
| | | Other | |
| Class | Detergent | Name | Performance |
| Polyoxyethylenes | Polyoxyethylenesorbitan | Tween 20 | Effective |
| (nonionic) | monolaurate | | |
| | Polyoxyethylenesorbitan | Tween 80 | Effective |
| | monooleate | | |
| | Octylphenoxy Poly(Ethyleneoxy) | Igepal CA | Effective |
| | ethanol | 630 | |
| | t-Octylphenoxypolyethoxyethanol | Triton | Effective |
| | | X100 | |
| | t-Octylphenoxypolyethoxyethanol | Triton | Effective |
| | | X114 | |
| | Sorbitan Monolaurate | Span 20 | Not effective |
| | Pluronic F-68 | N/A | Insoluble |
| Quaternary | Hexadecyltrimethylammonium | CTAB | Effective |
| ammonium | bromide | | |
| compounds | Dodecyltrimethylammonium | N/A | Effective |
| (cationic) | bromide | | |
| | Ethylhexadecyldimethylammonium | N/A | Effective |
| | bromide | | |
| | Benzethonium chloride | Hyamine | Effective |
| | | 1622 | |
| | Benzyldimethylhexadecylammonium | N/A | Less effective |
| | chloride | | |
| | Decamethonium bromide | N/A | Not effective |
| | Dimethyldioctadecylammonium | N/A | Insoluble |
| | bromide | | |
| Alkyl | N-Octyl-B-D-thioglucopyranoside | OTG | Not effective |
| thioglucosides | | | |
| (nonionic) | | | |
| Big Chap Series | [N,N′-Bis (3-D-gluconamidopropyl) | BIG CHAP | Not effective |
| (nonionic) | cholamide] | | |
| Glucamides | Decanoyl-N-methylglucamide | MEGA-10 | Not effective |
| (nonionic) | | | |
| Digitonin | Digitonin | Digitin | Not effective |
| (nonionic) | | | |
| Saponin | Saponin | N/A | Not effective |
| (nonionic) | | | |
| Betaines | N-Tetradecyl-N,N-dimethyl-3- | SB3-14 | Not effective |
| (zwitterionic) | ammonio-1-propanesulfate | | |
| Chaps Series | (3-[3-Cholamidopropyl) | CHAPSO | Not effective |
| (zwitterionic) | dimethylammonio]-1- | | |
| | propanesulfonate | | |
| Alkyl sulfates | Sodium Dodecyl Sulfate | SDS | Insoluble |
| (anionic) | | | |
| Bile Acids | Sodium Deoxycholate | N/A | Insoluble |
| (anionic) | | | |

Example 2 Summary: This example illustrates the effectiveness of various detergents in purifying RNA from difficult plant tissues.

### EXAMPLE 3

### Effects of LiCl Concentration on RNA Binding from Plant Tissue Extract to Silica Matrix

Pine needles and corn leaves were each ground to a fine powder in liquid nitrogen. For each assay, 100 mg of powdered plant material was lysed at 56° C. for 3 minutes in 500 μl of a lysis solution containing 6 M guanidine hydrochloride, 50 mM Tris-HCl, 95 mM EDTA, 1% Tween 20, 1% 2-mercaptoethanol, pH 7.8. Bulk cellular debris was removed by centrifugation for 3 minutes at 16,000×g. The supernatant extract was filtered through a filtration column (Sigma Number C6866) by centrifugation for 1 minute at 16,000×g to remove residual cellular debris. The clarified extract was mixed with a half volume of one of the five binding solutions comprising 8, 9, 10, 11, and 12 M LiCl, respectively. The combinations resulted in a series of LiCl concentrations ranging from 2.7 and 4 M in the binding mixture. RNA binding, washing, and elution were carried out as described in Example 2. Purified RNA was analyzed by a spectrophotometer and agarose gel electrophoresis.

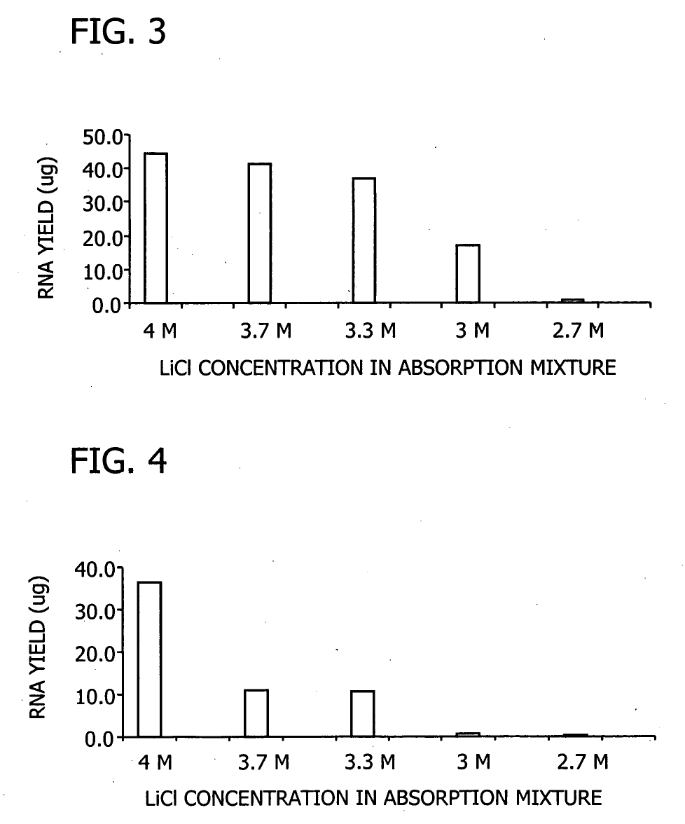

Results of spectrophotometric analysis: The results are shown in FIGS. 3 and 4. Very little RNA was recovered when the LiCl concentration in the binding mixture was less than 3 M. RNA recovery increased as the LiCl concentration in the binding mixture increased. The A₂₆₀/A₂₈₀ ratios of RNA samples purified with greater than 3 M of LiCl were between 2.0 and 2.2.

Results of agarose gel analysis: RNA integrity was confirmed in all RNA samples purified with greater than 3 M LiCl, with the 25 S and 18 S ribosomal RNAs appearing as discrete bands and in approximately 2:1 ratio.

Example 3 Summary: This example illustrates the significance of a LiCl concentration of at least about 3 M or more in effectively binding RNA from plant extract to a silica matrix. The results suggest that the RNA binding mechanism is different from that of RNA precipitation by LiCl.

### EXAMPLE 4

### Effects of Different Guanidine Salts on RNA Purification from Different Plant Tissues

Pine needles, grape leaves, and corn leaves were each ground to a fine powder in liquid nitrogen. For each assay, 100 mg of powdered plant material was lysed for 3 minutes at 56° C. in one of the four lysis solutions: (A) 6 M guanidine hydrochloride, 50 mM Tris-HCl, 90 mM EDTA, 1.5% Tween 20, 1% 2-mercaptoethanol, pH 7.5; (B) 6 M guanidine hydrochloride, 50 mM Tris-HCl, 90 mM EDTA, 1.5% CTAB, 1% 2-mercaptoethanol, pH 7.5; (C) 4 M guanidine thiocyanate, 50 mM Tris-HCl, 90 mM EDTA, 1.5% Tween 20, 1% 2-mercaptoethanol, pH 7.5; (D) 4 M guanidine thiocyanate, 50 mM Tris-HCl, 90 mM EDTA, 1.5% CTAB, 1% 2-mercaptoethanol, pH 7.5. Bulk cellular debris was removed by centrifugation for 3 minutes at 16,000×g. The supernatant extract was filtered through a filtration column (Sigma Number C6866) by centrifugation for 1 minute at 16,000×g to remove residual cellular debris. The clarified lysate was mixed with 250 μl of one of the two binding solutions: 1) 12 M LiCl; 2) 100% ethanol. Samples lysed with Lysis Solutions A and B were mixed with 12 M LiCl. Samples lysed with Lysis Solutions C and D were mixed with 12 M LiCl and 100% ethanol, respectively. The binding mixture was forced through a silica binding column (Sigma Number C6991) by centrifugation for 1 minute at 16,000×g. RNA washing and elution were conducted as described in Example 2. Purified RNA was analyzed by a spectrophotometer and by agarose gel electrophoresis.

Results of Spectrophotometric Analysis of Pine Needles
(a) Lysis Solution A yielded 27 μg of RNA and an A₂₆₀/A₂₈₀ ratio of 2.1.
(b) Lysis Solution B yielded 21 μg of RNA and an A₂₆₀/A₂₈₀ ratio of 2.2.
(c) Lysis Solutions C and D yielded no RNA, regardless of whether 12 M LiCl or ethanol was used as binding solution.

The results illustrate that guanidine hydrochloride is effective, but guanidine thiocyanate is ineffective for RNA purification from pine needles regardless of what type of detergent (nonionic or cationic) or what type of binding solution (12 M LiCl or ethanol) is used.

Results of Spectrophotometric Analysis of Grape Leaves
(a) Lysis Solution A yielded 31 μg of RNA and an A₂₆₀/A₂₈₀ ratio of 2.2.
(b) Lysis Solution B yielded 23 μg of RNA and an A₂₆₀/A₂₈₀ ratio of 2.2.
(c) Lysis Solutions C and D yielded no RNA, regardless of whether 12 M LiCl or ethanol was used as binding solution.

The results illustrate that guanidine hydrochloride is effective, but guanidine thiocyanate is ineffective for RNA purification from grape leaves regardless of what type of detergent (nonionic or cationic) or what type of binding solution (12 M LiCl or ethanol) is used.

Results of Spectrophotometric Analysis of Corn Leaves
(a) Lysis Solution A yielded 48 μg of RNA and an A₂₆₀/A₂₈₀ ratio of 2.2.
(b) Lysis Solution B yielded 49 μg of RNA and an A₂₆₀/A₂₈₀ ratio of 2.2.
(c) Lysis Solution C yielded 53 μg of RNA and an A₂₆₀/A₂₈₀ ratio of 2.2 when ethanol was used as binding solution, but it yielded no RNA when 12 M LiCl was used as binding solution.
(d) Lysis Solution D yielded 47 μg of RNA and an A₂₆₀/A₂₈₀ ratio of 2.2 when ethanol was used as binding solution, but it yielded no RNA when 12 M LiCl was used as binding solution.

The results show that with the non-difficult corn leaf tissue guanidine thiocyanate is effective when ethanol is used as binding solution. Corn tissues do not require a detergent in RNA purification (data not shown).

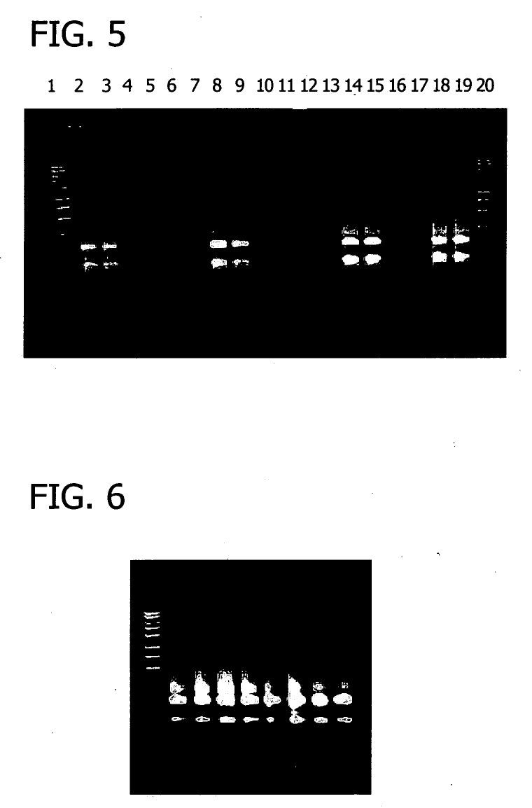

Results of agarose gel analysis: Pine needle and grape leaf RNA samples were analyzed with 2 μl of eluate and corn leaf RNA samples were analyzed with 1 μl of eluate in 1% nondenaturing agarose gel. The results are shown in FIG. 5.

Lanes 1 and 20: 1 kb DNA ladder; lane 2: pine needle by Lysis Solution A; lane 3: pine needle by Lysis Solution B; lane 4: pine needle by Lysis Solution C and 12 M LiCl; lane 5: pine needle by Lysis Solution D and 12 M LiCl; lane 6: pine needle by Lysis Solution C and ethanol; lane 7: pine needle by Lysis Solution D and ethanol; lane 8: grape leaf by Lysis Solution A; lane 9: grape leaf by Lysis Solution B; lane 10: grape leaf by Lysis Solution C and 12 M LiCl; lane 11: grape leaf by Lysis Solution D and 12 M LiCl; lane 12: grape leaf by Lysis Solution C and ethanol; lane 13: grape leaf by Lysis Solution D and ethanol; lane 14: corn leaf by Lysis Solution A; lane 15: corn leaf by Lysis Solution B; lane 16: corn leaf by Lysis Solution C and 12 M LiCl; lane 17: corn leaf by Lysis Solution D and 12 M LiCl; lane 18: corn leaf by Lysis Solution C and ethanol; lane 19: corn leaf by Lysis Solution D and ethanol.

Example 4 Summary: This example illustrates the effectiveness of the combination of guanidine hydrochloride and a detergent for RNA purification from difficult plant tissues (pine needles and grape leaves). The combination of guanidine thiocyanate and a detergent is not effective for difficult plant tissues regardless of what is used as binding solution.

### EXAMPLE 5

### Effects of 2-Mercaptoethanol on RNA Purification from Plant Tissues

Pine needles and tomato leaves were each ground to a fine powder in liquid nitrogen. For each assay, 100 mg of powdered plant material was lysed for 3 minutes at 56° C. in one of the two lysis solutions: (A) 6 M guanidine hydrochloride, 50 mM Tris-HCl, 90 mM EDTA, 1.5% Tween 20, pH 7.5; (B) 6 M guanidine hydrochloride, 50 mM Tris-HCl, 90 mM EDTA, 1.5% Tween 20, 1% 2-mercaptoethanol, pH 7.5. Removal of cellular debris, RNA binding, washing, and elution were carried out as described in Example 2. Purified RNA was analyzed by a spectrophotometer and by agarose gel electrophoresis.

Results of Spectrophotometric Analysis of Tomato Leaf
(a) Lysis Solution A yielded 150 and 165 μg of RNA per sample and an A₂₆₀/A₂₈₀ ratio of 2.2.
(b) Lysis Solution B yielded 153 and 172 μg of RNA per sample and an A₂₆₀/A₂₈₀ ratio of 2.2.

Results of Spectrophotometric Analysis of Pine Needle
(a) Lysis Solution A yielded 62 and 77 μg of RNA per sample and an A₂₆₀/A₂₈₀ ratio of 2.2.
(b) Lysis Solution B yielded 65 and 75 μg of RNA per sample and an A₂₆₀/A₂₈₀ ratio of 2.2.

The results illustrate that there was no significant difference in RNA yield or quality ratio with or without 2-mercaptoethanol.

Results of agarose gel analysis: Pine needle RNA samples were analyzed with 1 μl of eluate and tomato leaf RNA samples were analyzed with 0.5 μl of eluate in 1% nondenaturing agarose gel. The results are shown in FIG. 6.

Lane 1: 1 kb DNA ladder; lanes 2 & 3: tomato leaf RNA samples purified without 2-mercaptoethanol; lanes 4 & 5: tomato leaf RNA samples purified with 2-mercaptoethanol; lanes 6 & 7: pine needle RNA samples purified without 2-mercaptoethanol; lanes 8 & 9: pine needle RNA samples purified with 2-mercaptoethanol.

Example 5 Summary: This example illustrates that the reducing agent 2-mercaptoethanol is not essential for RNA purification using the present invention.

### EXAMPLE 6

### Purification of RNA from Seed and Tuber

Canola seed, corn seed, and potato tuber were each ground to a fine powder in liquid nitrogen. For each assay, 100 mg of powdered plant material was lysed for 3 minutes at 56° C. (canola seed) or at room temperature (corn seed and potato tuber) in a lysis solution containing 6 M guanidine hydrochloride, 50 mM Tris-HCl, 90 mM EDTA, 1.5% Tween 20, 1% 2-mercaptoethanol, pH 7.5. Removal of cellular debris, RNA binding, washing, and elution were carried out as described in Example 2. Purified RNA was analyzed by a spectrophotometer and by agarose gel electrophoresis.

Results of Spectrophotometric Analysis:

(1) Potato tuber: RNA Yield was 19 μg; A₂₆₀/A₂₈₀ ratio was 1.9.

(2) Corn seed: RNA yield was 23 μg; A₂₆₀/A₂₈₀ ratio was 2.2.

(3) Canola seed: RNA yield was 76 μg; A₂₆₀/A₂₈₀ ratio was 2.2.

Results of agarose gel electrophoresis: RNA integrity was confirmed in all samples, with the 25 S and 18 S ribosomal RNAs appearing as discrete bands and in approximately 2:1 ratio. No genomic DNA was detectable on the gel.

Example 6 Summary: This example illustrates that the method of the present invention is also suitable for RNA purification from seeds and tuber, which are enriched with carbohydrates (corn seed and potato tuber) or lipids (canola seed).

### EXAMPLE 7

### Purification of RNA from Animal Sources

RNA purification from HeLa cells

HeLa cells cultured in DMEM medium with 10% FBS were harvested at close to 100% confluence. Cells were washed with Hank balanced salt solution, detached with Trypsin EDTA solution, and resuspended in culture medium. Aliquots of 3 million cells each were prepared in 2-ml micro-centrifuge tubes and culture medium was removed by centrifugation. For each RNA purification, 3 million HeLa cells were lysed for 3 minutes at room temperature in 250 μl of a lysis solution containing 6 M guanidine hydrochloride, 50 mM Tris-HCl, 90 mM EDTA, 1% Tween 20, 1% 2-mercaptoethanol, pH 7.5. Lysate was filtered through a filtration column (Sigma Number C6866) by centrifugation at 16,000×g for 1 minute. The clarified lysate was then mixed with 370 μl of a 12 M LiCl binding solution and the mixture was forced through a silica binding column (Sigma Number C6991) by centrifugation at 16,000×g for 1 minute. The column was washed once with 500 μL of a 2 M LiCl solution with 1 minute of centrifugation at 16,000×g, and then twice with 500 μl of an alcohol solution (80% ethanol, 10 mM Tris, pH 7.0), by centrifugation at 16,000×g for 30 seconds. After the column was dried by centrifugation at 16,000×g for 1 minute, bound RNA was eluted in 50 μl of RNase-free water by centrifugation at 16,000×g for 1 minute. The elution was repeated once. Purified RNA was analyzed by a spectrophotometer and by agarose gel electrophoresis.

RNA Purification from Mouse Spleen

For each RNA purification, 40 mg of mouse spleen tissue was homogenized with a Brinkman Polytron PT 1200 in 500 μl of a lysis solution containing 6 M guanidine hydrochloride, 50 mM Tris-HCl, 90 mM EDTA, 1% Tween 20, 1% 2-mercaptoethanol, pH 7.5. Lysate was filtered through a filtration column (Sigma Number G6415). The clarified lysate was then mixed with 500 μl of a 12 M LiCl binding solution and the mixture was forced through a silica binding column (Sigma Number G4669) by centrifugation at 16,000×g for 1 minute. The column was washed once with 500 μl of a 2 M LiCl solution by centrifugation at 16,000×g for 1 minute, and then twice with 500 μl of an alcohol solution (80% ethanol, 10 mM Tris, pH 7.0), by centrifugation at 16,000×g for 30 seconds. After the column was dried by centrifugation at 16,000×g for 1 minute, bound RNA was eluted in 50 μl of RNase-free water by centrifugation at 16,000×g for 1 minute. The elution was repeated once. Purified RNA was analyzed by a spectrophotometer and by agarose gel electrophoresis.

Comparative example of RNA purification from animal sources

A commercial kit (GenElute Mammalian Total RNA Kit, manufactured by Sigma) was used to extract RNA from the same sources of animal material. Each RNA extraction used 3 millions of HeLa cells or 40 mg of mouse spleen tissue. RNA purification was carried out according to the kit's instruction. Purified RNA was analyzed by a spectrophotometer and by agarose gel electrophoresis.

Results of Spectrophotometric Analysis:
(1) HeLa cells: The present invention yielded 94 μg of RNA and an A₂₆₀/A₂₈₀ ratio of 2.1. The GenElute Mammalian Total RNA Kit yielded 105 μg of RNA and an A₂₆₀/A₂₈₀ ratio of 2.1.
(2) Mouse spleen: The present invention yielded 210 μg of RNA and an A₂₆₀/A₂₈₀ ratio of 2.1. The GenElute Mammalian Total RNA Kit yielded 203 μg of RNA and an A₂₆₀/A₂₈₀ ratio of 2.1.

Results of agarose gel analysis: RNA integrity was confirmed in all samples, with the 28 S and 18 S ribosomal RNAs appearing as discrete bands and in approximately 2:1 ratio. Agarose gel analysis revealed that RNA samples prepared by the GenElute Mammalian Total RNA Kit contained more genomic DNA than RNA samples prepared by the present invention.

Example 7 Summary: This example illustrates that the present invention can be effectively applied to animal sources in the isolation of RNA.

### EXAMPLE 8

### RNA Binding to Silica Matrix with and without Chaotrope

Tomato total RNA was prepared by lysing 100 mg of powdered tomato leaf tissue at 56° C. for 3 minutes in a lysis solution containing 6 M guanidine hydrochloride, 50 mM Tris-HCl, 90 mM EDTA, 1.5% Tween 20, 1% 2-mercaptoethanol, pH 7.5. Removal of cellular debris, RNA binding, washing, and elution were carried out as described in Example 2. Purified RNA was quantified by a spectrophotometer. Multiple RNA samples were pooled and diluted in RNA-free water to 1 μg/μl. For each binding assay, 50 μl of the RNA sample (50 μg) was combined with 450 μl of one of the two solutions: 1) 50 mM Tri-HCl, 90 mM EDTA, 1.5% Tween 20, pH 7.5; 2) 6 M guanidine hydrochloride, 50 mM Tri-HCl, 90 mM EDTA, 1.5% Tween 20, pH 7.5). The sample was then mixed with 250 μl or 500 μl of a 12 M LiCl binding solution to a final concentration of 4 M or 6 M LiCl. The mixture was then forced through a silica binding column (Sigma Product Number C6991) by centrifugation for 1 minute at 16,000×g. The column was washed twice, each with 500 μl of an alcohol solution (80% ethanol, 10 mM Tris, pH 7.0), by centrifugation for 30 seconds at 16,000×g. After the column was dried by centrifugation for 1 minute at 16,000×g, bound RNA was eluted in 50 μl of RNase-free water by centrifugation for 1 minute at 16,000×g. The elution was repeated once. Recovered RNA samples were analyzed by a spectrophotometer and by agarose gel electrophoresis.

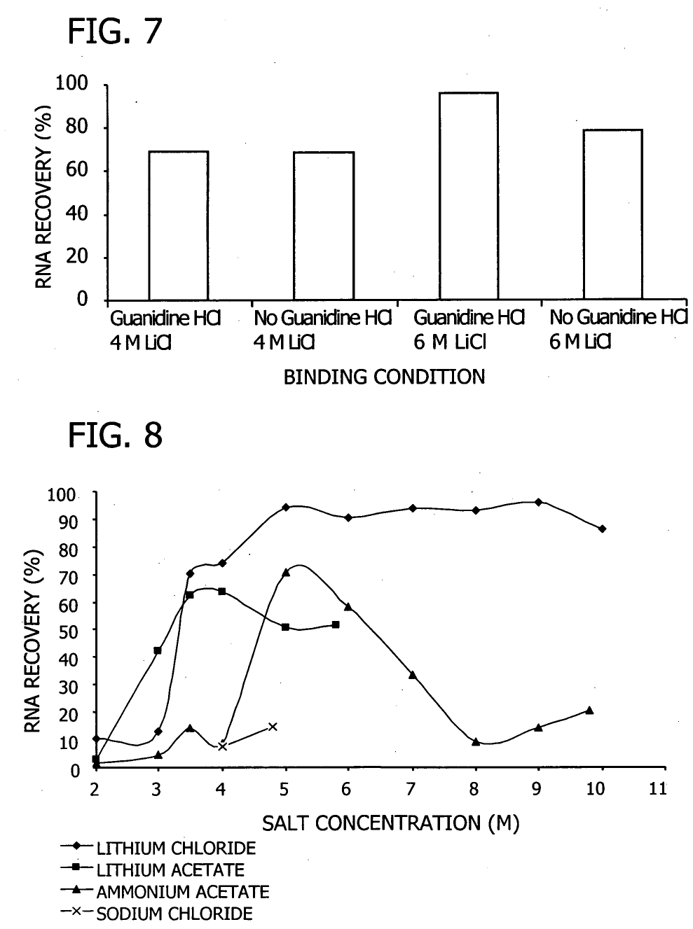

Results of spectrophotometric analysis: The results are shown in FIG. 7. The results show that there was no significant difference in RNA binding to silica matrix by LiCl with or without guanidine hydrochloride.

Results of agarose gel analysis: The integrity of recovered RNA was confirmed in all samples, with the 25 S and 18 S ribosomal RNAs appearing as discrete bands and in approximately 2:1 ratio.

Example 8 Summary: This example illustrates that LiCl is an effective binding agent for binding RNA from a RNA-containing solution to silica matrix with or without guanidine hydrochloride.

### EXAMPLE 9

### Comparison of Monovalent Salts on RNA Binding to Silica Matrix

For each assay, 50 μl of tomato leaf RNA sample (50 μg) prepared by the method described in Example 8 was brought up to a total of 750 μl with water and one of the four salt solutions: 1) 12 M LiCl; 2) 6 M lithium acetate; 3) 10 M ammonium acetate; 4) 5 M NaCl, to obtain a desired final salt concentration in the binding mixture. The mixture was then forced through a binding column (Sigma Product Number C6991) by centrifugation for 1 minute at 16,000×g. The column was washed twice, each with 500 μl of an alcohol solution (80% ethanol, 10 mM Tris, pH 7.0), by centrifugation for 30 seconds at 16,000×g. After the column was dried by centrifugation for 1 minute at 16,000×g, bound RNA was eluted in 50 μl of RNase-free water by centrifugation for 1 minute at 16,000×g. The elution was repeated once. Recovered RNA samples were analyzed by a spectrophotometer and by agarose gel electrophoresis.

Results of spectrophotometric analysis: The results are shown in FIG. 8. The results illustrates that LiCl is the most effective monovalent salt in effecting RNA binding to silica matrix. Lithium acetate and ammonium acetate are also effective, though to a lesser degree, at certain concentration regimes. NaCl is ineffective for the RNA binding.

Results of agarose gel analysis: The integrity of recovered RNA was confirmed in all samples, with the 25 S and 18 S ribosomal RNAs appearing as discrete bands and in approximately 2:1 ratio.

Example 9 Summary: This example illustrates the effectiveness of LiCl as a monovalent salt binding agent for RNA binding. This example also illustrates the effectiveness of other salts as binding agents.

### EXAMPLE 10

### RNA Binding on Siliceous and Non-Siliceous Matrices

For each assay, 50 μl of tomato leaf RNA sample (50 μg) prepared by the method described in Example 8 was combined with 450 μl of a Tris-EDTA buffer (50 mM Tri-HCl, 90 mM EDTA, pH 7.9). The sample was then mixed with 360 μl of a 12 M LiCl binding solution and the final LiCl concentration in the binding mixture was 5 M. The mixture was then forced through a binding column by centrifugation at 16,000×g for 1 minute. Four different types of binding matrix were tested: 1) silica filter; 2) silica filter with latex binder; 3) acrylic copolymer; 4) polytetrafluoroethylene. Each binding column contained 3 layers of one of the four binding matrices. The first three types of matrix are highly hydrophilic, and the last matrix is highly hydrophobic. The column was washed twice, each with 500 μl of an alcohol solution (80% ethanol, 10 mM Tris, pH 7.0), by centrifugation for 30 seconds at 16,000×g. After the column was dried by centrifugation for 1 minute at 16,000×g, bound RNA was eluted in 50 μl of RNase-free water by centrifugation for 1 minute at 16,000×g. The elution was repeated once. Recovered RNA samples were analyzed by a spectrophotometer and by agarose gel electrophoresis.

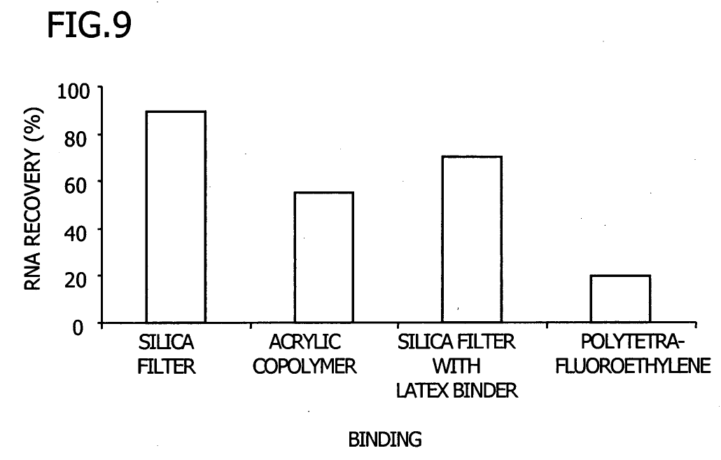

Results of spectrophotometric analysis: The results are shown in FIG. 9. The results illustrate that a silica filter is the most efficient matrix for RNA binding by LiCl, followed by a silica filter with latex binder. The acrylic copolymer matrix also bound a significant amount of input RNA (>50%) under the same condition, while the hydrophobic polytetrafluoroethylene matrix is ineffective, capturing less than 20% of input RNA under the same condition.

Results of agarose gel analysis: RNA integrity was confirmed in all recovered RNA samples, with the 25 S and 18 S ribosomal RNAs appearing as discrete bands and in approximately 2:1 ratio.

Example 9 Summary: This example illustrates the effectiveness of various hydrophilic binding matrices in binding RNA.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained.

As various changes could be made in the above methods and products without departing from the scope of the invention, it is intended that all matter contained in the above description and shown in any accompanying drawings shall be interpreted as illustrative and not in a limiting sense.

What is claimed is:

1. A method of isolating RNA from a biological sample, said method comprising:
a) lysing the biological sample with a solution comprising a chaotrope and a detergent to release RNA into the solution; and
b) binding the released RNA to a matrix in the presence of a monovalent salt, wherein the monovalent salt concentration in the RNA-containing solution is at least about 3 M.

2. The method of claim 1, wherein the chaotrope is guanidine hydrochloride.

3. The method of claim 1, wherein the detergent is a nonionic polyoxyethylene compound.

4. The method of claim 3, wherein the nonionic polyoxyethylene compound is selected from the group consisting of polyoxyethylenesorbitan monolaurate, polyoxyethylenesorbitan monooleate, octylphenoxy poly(ethyleneoxy)ethanol, t-octylphenoxypolyethoxyethanol, and Nonidet P-40.

5. The method of claim 1, wherein the detergent is a cationic quaternary ammonium compound.

6. The method of claim 5, wherein the cationic quaternary ammonium compound is selected from the group consisting of CTAB, dodecyltrimethylammonium bromide, ethylhexadecyldimethylammonium bromide, benzethonium chloride, and benzyldimethylhexadecylammonium chloride.

7. The method of claim 1, wherein the monovalent salt is selected from the group consisting of lithium chloride, lithium acetate, and ammonium acetate.

8. The method of claim 7, wherein the monovalent salt is LiCl.

9. The method of claim 1, wherein the lysis solution further comprises a chelating agent.

10. The method of claim 9, wherein the chelating agent is selected from the group consisting of ethylenediaminetetraacetic acid, ethylene glycol bis(2-aminoethyl ether)-N,N,N′N′-tetraacetic acid, and cyclohexane-trans-1,2-diamine tetraacetic acid.

11. The method of claim 1, wherein the lysis solution further comprises a reducing agent.

12. The method of claim 11, wherein the reducing agent is selected from the group consisting of 2-mercaptoethanol and dithiothreitol.

13. The method of claim 1, wherein the released RNA is bound to the matrix in the presence of an alcohol.

14. The method of claim 13 wherein the alcohol is selected from the group consisting of ethanol and isopropanol.

15. The method of claim 1, wherein the matrix is a hydrophilic matrix.

16. The method of claim 15, wherein the hydrophilic matrix is an inorganic binding matrix.

17. The method of claim 16, wherein the inorganic binding matrix is selected from the group consisting of silica, diatomaceous earth, aluminum oxides, glass, titanium oxides, zirconium oxides, and hydroxyapatite.

18. The method of claim 16, wherein the inorganic binding matrix is a siliceous material.

19. The method of claim 15, wherein the hydrophilic matrix is an organic binding matrix.

20. The method of claim 19, wherein the organic binding matrix is selected from the group consisting of acrylic copolymer, cellulose, dextran, agarose, and acrylic amide.

21. The method of claim 1, wherein the RNA bound to the matrix is washed with a salt solution.

22. The method of claim 21, wherein the salt solution is selected from the group consisting of lithium chloride, guanidine thiocyanate, and guanidine hydrochloride.

23. The method of claim 1, wherein the RNA bound to the matrix is washed with an alcohol solution.

24. The method of claim 23, wherein the alcohol solution is selected from the group consisting of ethanol and isopropanol.

25. The method of claim 1 wherein the RNA bound to the matrix is eluted using an elution solution selected from the group consisting of RNase-free water and RNase-free low salt solution.

26. The method of claim 1, wherein the biological sample is plant tissue or cells.

27. The method of claim 1, wherein the biological sample is animal tissue or cells.

28. The method of claim 26, wherein the plant tissue comprises at least 0.05% phenolic or polyphenolic compounds.

29. The method of claim 26, wherein the plant tissue comprises at least 5% polysaccharides.

30. A method of isolating RNA from an RNA-containing solution, said method comprising:
binding the RNA to a matrix in the presence of a monovalent salt, wherein the monovalent salt in the RNA-containing solution has a concentration of at least about 3 M; and
separating the solution from the bound RNA.

31. The method of claim 30, wherein the monovalent salt is selected from the group consisting of lithium chloride, lithium acetate, and ammonium acetate.

32. The method of claim 31, wherein the monovalent salt is lithium chloride.

33. The method of claim 30, wherein the RNA-containing solution is mixed with a binding solution comprising a monovalent salt and a chaotrope.

34. The method of claim 33, wherein the monovalent salt is selected from the group consisting of lithium chloride, lithium acetate, and ammonium acetate.

35. The method of claim 33, wherein the chaotrope comprises guanidine hydrochloride.

36. The method of claim 30, wherein the matrix is a hydrophilic matrix.

37. The method of claim 36, wherein the hydrophilic matrix is an inorganic binding matrix.

38. The method of claim 37, wherein the inorganic binding matrix is selected from the group consisting of silica, diatomaceous earth, aluminum oxides, glass, titanium oxides, zirconium oxides, and hydroxyapatite.

39. The method of claim 36, wherein the inorganic binding matrix is a siliceous material.

40. The method of claim 36, wherein the hydrophilic matrix is an organic binding matrix.

41. The method of claim 40, wherein the organic binding matrix is selected from the group consisting of acrylic copolymer, cellulose, dextran, agarose, and acrylic amide.

42. The method of claim 30, wherein the bound RNA is washed with a wash solution selected from the group consisting of a salt solution and an alcohol solution.

43. The method of claim 42, wherein the salt solution is selected from the group consisting of lithium chloride, guanidine thiocyanate, and guanidine hydrochloride.

44. The method of claim 42, wherein the alcohol solution is selected from the group consisting of ethanol and isopropanol.

45. The method of claim 30, wherein the bound RNA is eluted with an elution solution selected from the group comprising RNase-free water and RNase-free low salt solution.

46. A reagent for isolating RNA from plant tissues or cells, said reagent comprising a detergent, a chaotrope, a chelator, and a reducing agent.

47. The reagent of claim 46, wherein the detergent is selected from the group consisting of nonionic polyoxyethylenes and cationic quaternary ammonium compounds.

48. The reagent of claim 46, wherein the chaotrope is guanidine hydrochloride.

49. The reagent of claim 46, wherein the chelator is selected from the group consisting of ethylenediaminetetraacetic acid, ethylene glycol bis(2-aminoethyl ether)-N,N,N′N′-tetraacetic acid, and cyclohexane-trans-1,2-diamine tetraacetic acid.

50. The reagent of claim 46, wherein the reducing agent is selected from the group consisting of 2-mercaptoethanol and dithiothreitol.

51. A kit for isolating RNA from a biological sample, said kit comprising:
a reagent comprising a chaotrope, a detergent, a chelator; and
a binding solution comprising at least about 3 M lithium chloride.

52. The kit of claim 51, wherein the reagent further comprises a reducing agent.

53. The kit of claim 51, wherein the kit further comprises a binding matrix wherein the binding matrix is selected from the group consisting of silica, diatomaceous earth, aluminum oxides, glass, titanium oxides, zirconium oxides, and hydroxyapatite.

54. A reagent for isolating RNA from plant tissues, said reagent comprising:
a detergent selected from the group consisting of nonionic polyoxyethylenes and cationic quaternary ammonium compounds;
guanidine hydrochloride; and
at least about 3 M lithium chloride.
